(19) United States
(12) Patent Application Publication
(10) Pub. No.: US 2006/0272434 A1
(43) Pub. Date: Dec. 7, 2006

(54) System and method of use for continuous deterioration measurements

(76) Inventors: Roumiana Petrova, New Milford, NJ (US); Naruemon Suwattananont, Kearny, NJ (US)

Correspondence Address: NEW JERSEY INSTITUTE OF TECHNOLOGY, 323 MARTIN LUTHER KING, JR. BOULEVARD, OFFICE OF RESEARCH AND DEVELOPMENT, NEWARK, NJ, 07102-1982, US

(21) Appl. No.: 11/446,090

(22) Filed: Jun. 2, 2006

Related U.S. Application Data
(60) Provisional application No. 60/687,181, filed on Jun. 3, 2005.

(51) Int. Cl. G01N 33/00

(52) U.S. Cl. 73/866

(57) ABSTRACT

A system and the method of use of the system for continuous deterioration rate measurements of sample materials are provided by the present invention. Mass loss is a versatile method for determining sample deterioration. Previous mass loss techniques employ determination at periodic intervals after removing the sample from the process environment thus providing non-continuous measurements, which affect accuracy and precision, increase analysis time, and are non-illustrative of actual working conditions. The disclosed invention suspends the sample, which is immersed in the process environment, from an electronic balance. The balance is connected to a computer for recording data on a predetermined time interval. The advantages of the disclosed continuous deterioration measurement system and method of use include, but are not limited to, an increase in measurement precision, simpler test method and calculations, versatility in sample material and process environment, and most importantly test conditions that simulate real working conditions.

### CROSS-REFERENCE TO RELATED APPLICATION

The present application claims the benefit of a co-pending, commonly assigned provisional patent application entitled “Apparatus and Method of Use For Continuous Deterioration Measurements,” which was filed on Jun. 3, 2005 and assigned Ser. No. 60/687,181. The contents of the foregoing provisional patent application are hereby incorporated by reference.

### FIELD OF THE INVENTION

The present invention relates to the field of deterioration testing. It more particularly relates to continuous measurements based on weight or mass loss of any material placed in a harsh or corrosive environment. More particularly, the present invention relates to the method and system used to provide continuous degradation measurements of metals, nonmetals, and composites thereof, both with and without coatings, when the metals or nonmetals are placed in a harsh or corrosive environment by measuring the weight or mass of the metal or nonmetal lost due to deterioration.

### BACKGROUND OF THE INVENTION

Corrosion is deterioration of a material resulting from chemical attack by the material's environment. Corrosion resistance or chemical resistance depends on many factors. A comprehensive study of corrosion requires a deep knowledge of several scientific fields. Thermodynamics and electrochemistry are of significant importance in understanding and controlling corrosion.

The corrosion of materials refers to the chemical attack of metals that occurs most by electrochemical reaction, since metals have free electrons which may set up electrochemical cells. Metallic materials can also corrode by direct chemical attack from chemical solutions. Corrosion is an electrochemical process which involves transfer of electronic charge. Corrosion consists of two different types of reactions, i.e. anodic and cathodic, occurring simultaneously. Anodic reaction involves loss of electrons or increase of the oxidation state of metal, while cathodic reaction involves the consuming the electrons.

Nonmetallic materials, such as polymers and ceramics, do not suffer electrochemical attack, but they can suffer from direct chemical attack.

The serious consequences of corrosion have become a problem of worldwide significance. Degradation and corrosion cause plant shutdowns, waste of valuable resources, reduction in efficiency, costly maintenance, and most importantly loss of safety and inhibition of technological progress. NACE International study shows the cost of corrosion in the USA is $276 billion/year.

Corrosion is an extremely destructive process and represents an enormous economic loss. Accordingly, engineers working in industry must be concerned about corrosion control and prevention.

The present disclosure applies to the deterioration of both metallic and nonmetallic materials. The term deterioration will be used throughout to generally reference the applicability of both the system and method of use of said system presently disclosed to both metallic and nonmetallic materials.

The most common technique to measure deterioration is the weight loss or mass loss technique. Weight loss and mass loss are used interchangeably in the present disclosure. Both mass and weight can be measured using this presently disclosed method and system. This method involves obtaining a sample or specimen of the material to be tested, which is known as a coupon. Generally the samples are prepared by cleaning the outer surfaces of debris so that the only measurable mass is the mass of the coupon. This is often done by using a grit sandpaper to grind off the dirt and debris then rinsing the coupon with acetone. The specimen preparation technique is outlined in ASTM G1-90 (Re-approved 1999), “Standard Practice for Preparing, Cleaning, and Evaluating Corrosion Test Specimens.”¹ The coupon is then exposed to the process environment for the specific duration of time. At the end of the delineated time period, the coupon is removed and cleaned to remove oxidation and debris that has formed. The coupon is then dried completely and weighed. The coupon is then returned to the process environment for the specified duration of time and then the process is repeated. The traditional mass loss technique is depicted in ASTM G31-72 (Re-approved 1999), “Standard Practice for Laboratory Immersion Corrosion Testing of Metals.”² Deterioration is commonly measured using the deterioration rate which is expressed as the mass loss over the period of time the coupon was exposed to the process environment.

There are many benefits to the mass loss technique. One benefit is that the coupons can be derived from almost any material, including most metal alloys, making it a very versatile method for many types of samples. A multitude of environments can be utilized for the process environment including gas, liquid and solid/particulate flow. Additionally, a visual inspection of the coupon's deterioration and oxidation deposits is possible. The measurements of mass and time and the calculations to determine the deterioration rate are simple. If there is localized deterioration this can be accounted for as well.

There are also many disadvantages to the traditional mass loss technique. The most significant disadvantage is that the measurements are disjointed rather than continuous. This can have a pronounced impact on the deterioration rate because if a deterioration upset occurs (this is when a piece of the coupon becomes separated from the remaining coupon sample) during the expose period, it is not possible to determine the precise time the deterioration upset took place under the traditional method, such as the ASTM method. Additionally dependant upon the peak values and the duration of exposure to the process environment, the mass loss may not be statistically significant. Also, when the sample is transferred between the process environment and the weighing stage, the coupon is exposed to a different environment that may affect the deterioration rate and not provide an accurate model of the deterioration under working conditions. Further, non-deteriorated material may be removed in the cleaning and drying process necessary for the traditional mass loss technique. Therefore, although traditional mass loss is a simple method to measure deterioration, it is a long process that requires extended time duration, advanced preparation and continuous attention.

The presently disclosed novel system and method of use of said system contain all of the same advantages present in the traditional mass loss technique but eliminate the associated disadvantages.

### SUMMARY OF THE INVENTION

According to the present disclosure, a continuous measurement based on mass loss of the coupon when it is placed in the harsh or corrosive process environment can be achieved by a novel experimental setup or system. In one embodiment, the coupon is suspended from a weighing device, such as a balance, into the process environment. During that time, the coupon undergoes constant and continual weighing. The mass measurements, which can be used for direct and immediate analysis of the raw data to generate, by way of example, the deterioration rate or graphical representations of the data, are transferred automatically to a recording device.

In one particular embodiment, the metal coupon is suspended via a platinum wire which is attached to the hook located on the underside of the electronic balance, as demonstrated in FIG. 1. An electonic balance is connected to a computer, and the continuous mass data is uploaded directly from the balance to the computer. The computer records the mass data and calcuates the mass as a function of time to generate a deterioration rate, which is illustrated as a graphical represention of mass and time.

In a further aspect of the present disclosure, the method of use of said system is designed and developed to evaluate the deterioration resistance of materials by measuring the loss of weight. The measurements taken at any time differential specified, including by way of example only, measurements taken every second, provide a continuous deterioration rate calculation. This continuous method, which eliminates the need for samples to be washed and then weighed at predetermined time intervals, contains many advantages. Numerous advantages result from the continuous weight loss measurements disclosed herein and the uses/applications thereof.

In a further aspect of the present disclosure, the temperature of the process environment can be altered as necessary to accommodate the system requirements. These requirements may be, by way of example only, based on the type of process environment, the concentration or strength of the process environment, the type of the sample undergoing deterioration, the length of time required for deterioration to be completed in accordance with the process environment and samples, or any combination of factors thereof.

For example, in exemplary embodiments of the present disclosure, the disclosed process environment can be any environment including gaseous environments, liquid environments and solid/particulate flow environments or any combination thereof. Conditions may vary within the process environment to generate multiple zones within a single process environment. By way of example only, there may be a process environment with two zones present simultaneously wherein one zone includes liquid components while the other zone includes gaseous components. The environments can be of any temperature and of any concentration or strength. By way of example, the process can be conducted at any temperature including room temperature or elevated temperatures. Similarly, by way of example only, the process environment can be a corrosive environment including various concentrations or strengths of HCl, KOH, or NaCl. Also, the process environment may be kept in a container, such as an Erlenmeyer flask. In the presently disclosed system and method, the process environment can be either a static or dynamic environment. In one embodiment where the process environment is static, there is minimal or no movement or current in the medium or of the sample. In another embodiment where the process environment is dynamic, the presently disclosed system and method can be utilized when either the process environment or the sample is in motion. The motion or movement can be caused by any force including, but not limited to, a magnetic stir mechanism, a rotary evaporator, a shaker, a mixer, an air flow, a fluid flow, natural phenomenon or the like.

For example only, in exemplary embodiments of the present disclosure, the sample undergoing deterioration rate analysis can be any material including, but not limited to, metal, metal alloys, ceramics, plastics, polycarbonate synthetic fibers, polymers, or natural materials, such as wood or cotton. Additionally, these samples can be coated or uncoated with most barrier or protective substances.

For example, in exemplary embodiments of the present disclosure, the manner in which the sample is suspended from the measuring device can vary greatly in both type and structural form. The medium for suspension can be any non-reactive entity. In one embodiment, the coupon or sample may be suspended using a suspension device which includes, but is not limited to a rod, a coil, a mesh, a chain, a string, a rope, a bar, or a wire. In some embodiments, the suspension device may be a stiff member. In alternate embodiments, the suspension device may be a flexible member. One illustrative embodiment is to use a platinum wire. Similarly, the sample can be attached to the non-reactive entity in anyway that will securely hold the sample. By way of example only, this may include wrapping the wire around the sample, creating a basket or creating a hook.

In one exemplary embodiment, the sample can be completely immersed in the process environment as suspended by the suspension device. The presently described method can also be used when the sample is buoyant. In one such embodiment a stiff member can be used to immerse the sample to calculate the deterioration rate. In another embodiment, the buoyant sample may span multiple zones within the process environment. Accordingly, deterioration measurements can be obtained for the one or more surfaces that are in contact with the desired zone or zones within the process environment.

For example, in exemplary embodiments of the present disclosure, the recording device can be any data recorder. By way of example, the data recorder could be a computer. If the measuring device were to be equipped with a data recorder such as an incorporated computer system, the external data recorder could be omitted from this disclosure. In an alternative embodiment, no recording device is necessary. In this embodiment, an individual can manually monitor the weighing device and record the data in a multitude of mediums including, by way of example, only a manual input into a computer or paper and pen.

For example, in exemplary embodiments of the present disclosure, the system conditions can also be altered in order to achieve varied deterioration or optimized analysis. By way of example only, these conditions may include, but are not limited to, alterations in temperature, time, process environment, pressure, moisture, recondensing of the process environment, or any variation or combination thereof.

For example, in exemplary embodiments of the present disclosure, the measuring device can be altered to be any weighing device. Electronic analytic balances are, by way of example only, one type of measuring device that would be sufficient for this process.

For example, in exemplary embodiments of the present disclosure, the weighing device used must include or be able to be adapted to include a means for attaching the suspension medium such that the weight of the sample is recorded by the weighing device. One such example is a hook located on the underside of an electric balance which is included in most commercial electric balances. Another embodiment would be to have a loop attached to the underside of the balance. Similarly, a permanently attached suspension device attached or affixed in lieu of the hook, which for example could be soldered to the underside or the top of an electric balance, would work as well.

These and other advantages, features and attributes of the continuous mass loss deterioration system and method for the present disclosure, and their advantageous applications and/or uses will be apparent from the detailed description which follows, particularly when read in conjunction with the figures appended hereto.

### BRIEF DESCRIPTION OF THE DRAWINGS

To assist those of ordinary skill in the relevant art in making and using the subject matter hereof, reference is made to the appended drawings, wherein:

FIG. 1 is a representation of one embodiment of the system or equipment or system set-up designed for continuous weighing of mass loss in a corrosive process environment.

FIG. 2 depicts the evaluation of a deterioration test of unboronized and boronized AISI 304 in 5% HCl, 10% HCl, 15% HCl (304P untreated stainless steel; 304B boronized stainless steel) using the continuous mass loss methodology according to the present disclosure.

FIG. 3 depicts the evaluation of a deterioration test of unboronized and boronized AISI 304 in 5% KOH (304P untreated stainless steel; 304B boronized stainless steel) using the continuous mass loss methodology according to the present disclosure.

FIG. 4 depicts the evaluation of a deterioration test of unboronized and boronized Inconel 625 in 15% HCl (Inconel 625P untreated Inconel; Inconel 625B boronized Inconel) using the continuous mass loss methodology according to the present disclosure.

### DETAILED DESCRIPTION OF THE INVENTION

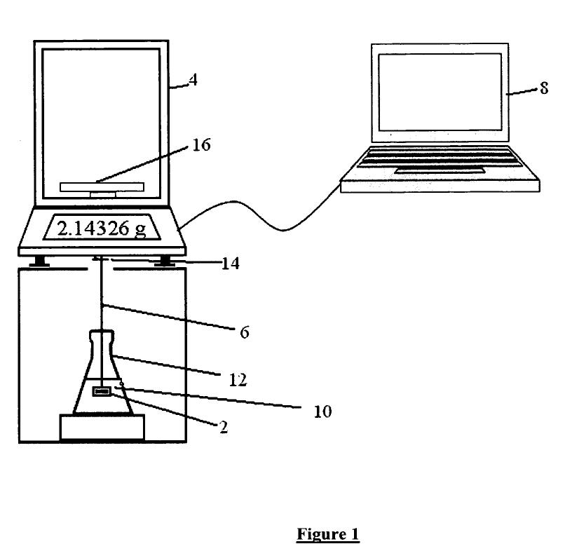

One embodiment of the continuous mass loss system [FIG. 1] which measures the deterioration of a sample coupon (2) consists of an electric analytical balance (4), a platinum wire (6) and a computer (8). The platinum wire (6) consists of a hook end and a coupon end. The coupon end of the platinum wire (6) is wound around the coupon (2) in a fashion that securely holds the coupon (2) and suspends it in the process environment (10) which is contained in an Erlenmeyer flask (12). The coupon (2) is suspended in the process environment (10) so that all surfaces of the coupon (2) are enveloped by the process environment (10). The hook end of the platinum wire (6) is attached to the hook (14) located on the underside of the electric analytic balance (4). The hook (14) is an integrated part of the commercially made electric analytic balance (4) which measures the weight of the coupon (2). This measurement is identical to the measurement that would be received if the sample had been placed on the scale surface (16).

The electric analytic balance (4), from which the sample coupon (2) is suspended, is connected to the computer (8) which is acting as a data recording device. The data generated from the deterioration of the sample coupon (2) which is detected by the electric analytic balance (4) is transferred to the computer (8) so that the information can be recorded and processed. The processing done by the computer (8) includes calculations of the deterioration rate as well as graphic representations of the deterioration.

In another embodiment, the continuous mass loss system [FIG. 1] incorporates a temperature control device. The temperature control device could either heat or cool the environment. In one such embodiment, the Erlenmeyer flask (12) would be placed on a heating device like a hotplate or in a water bath.

In an embodiment of the method of use of the continuous mass loss system [FIG. 1], the system measures the deterioration of the sample coupon (2). Deterioration rate is the calculation of mass as a function of time. The method employed in the present disclosure consists of the electronic analytic balance (4) measuring the mass of the sample coupon (2) every second. The computer (8) records the mass as measured by the electronic analytic balance (4) and then calculates deterioration rate. The computer (8) also generates graphical representations of the deterioration rate and retains the generated and the calculated figures for further use.

The continuous mass loss measurement system and method of use for said system of the present disclosure offer significant advantages relative to prior art methods for deterioration testing. The advantageous properties and/or characteristics of the disclosed continuous mass loss measurement system and method of use include, but are not limited to, an increase in precision of the measurements taken, simpler test method and calculations, versatility in sample material and process environment, and most importantly test conditions that simulate real working conditions.

First, the presently disclosed system and method of use of said system provide more precise measurements. This outcome is a direct result of the ability to obtain results at any specified time differential, including by way of example only, on a second by second basis. This allows precise determinations of the rate of deterioration and specific identification of the moment of deterioration upset which in turn prevents an accurate indication of the true deterioration rate. Precision is only limited by the sensitivity of the measuring instrument or device. In one embodiment of the presently disclosed method, the weight loss and the eventual calculation of the deterioration rate maintains a ±0.00001 g sensitivity. In contrast to the ASTM G-31-72 method, the sensitivity is limited to ±0.0005 g.²

Second, the presently disclosed system and method of use creates an environment of constant chemical conditions. This is a vital aspect in that the consistent environment closely mimics real working conditions. This in turn also provides a more valid calculation of deterioration rate under conditions that will be present in the laboratory or field.

The third advantage of the present disclosure is that the oxidation layer or deterioration products remain intact on the coupon. This is in direct contrast to the traditional mass loss method where the tested samples must be washed. In the traditional method, the washing of the coupon eliminates all oxidation products which formed on the material's surfaces. This alters the accuracy of the deterioration rate by altering surface conditions. The removal of the oxidation layer may alter the rate of the deterioration by either increasing or decreasing the rate. Moreover, depending on many factors, predicting how the factors affected the rate would be difficult if not impossible; therefore, the resulting deterioration rate would be imprecise.

Fourth, the kinetic parameters are easier to calculate. Simpler calculations directly impact the accuracy of the deterioration rate by introducing less variables and assumptions. This also quickens the analysis process.

Fifth, the presently disclosed system and method of use of said system substantially decreases the time required to perform deterioration testing and evaluation. The traditional steps of removing the coupon from the process environment, washing the coupon, drying the coupon and weighing the coupon, substantially increase the time necessary for proper analysis. According to ASTM G31-72, the most common test durations are 48 to 168 hours.² If the testing requires the coupon to be exposed to the process environment for 48 hours, the testing will take substantially more than 48 hours to account for the washing, drying and weighing steps. However under the method presently disclosed the testing can be completed in the 48 hours in which the coupon is to be exposed to the process environment.

Sixth, under the presently disclosed method, the coupon can be virtually any shape, size, or weight. This provides increased versatility of the method. Under the traditional method there were more stringent shape, size and weight requirements due to the surface area requirements, as outlined in ASTM G31-72.²

Seventh, under the present disclosure, the mass loss can be measured every second or any selected time interval, which affords more precise measurement and deterioration rate calculations.

Eighth, since all the data is generated by the electronic balance and the information is then sent directly to the attached computer, this decreases human error as well as decreases time in the analysis process. The data is stored in the computer and is readily available for calculations, graphical representation, or other means of analysis.

Finally, the presently disclosed system is dramatically simpler than the apparatus described in ASTM G-31-72.² The disclosed system can accommodate any experimental set up ranging from an open beaker to the typical resin flask disclosed in the ASTM standard. The reflux condenser and associated apparatus are not necessary but can be incorporated into the presently disclosed system as long as the coupon can be suspended from the measuring device. The inclusion of a reflux condenser can be of assistance when the system or method is being used when the process environment is at an increased temperature.

An exemplary embodiment of the system and method of use presently disclosed of illustrates the need for such an system and method of use of said system. This exemplary embodiment is the analysis of coupons containing a protective coating. One specific exemplary embodiment of such a coating would be a boronizing coating placed on coupons which are specimens consisting of AISI 1018, AISI 4340, AISI 304, Inconel 625, and Tantalum (99.98% pure). These coupons were 10×10×3 mm in dimension. The metal and alloy specimens were grinded on 120, 220, 400, and 600 grit sand paper and cleaned with acetone before boronizing. Originally the powder packing method with B₄C, KBF₄ and Al₂O₃ powders was used to boronize specimens at the different heat treatment temperature depending on the types of substrates. Specimens of AISI 1018, AISI 4340 and AISI 304 were boronized at 850° C. for 4 hours, Inconel 625 at 950° C. for 4 hours and Tantalum at 1050° C. for 8 hours. The prepared coupons underwent analysis according to the continuous mass loss method as presently disclosed to investigate the deterioration resistance of boronized and unboronized specimens. The presently disclosed system or set-up equipment for the continuous weighing method is illustrated in FIG. 1. In this exemplary embodiment, the specimen coupons were tested in 5%, 10%, and 15% HCl, 5% KOH, and 4% NaCl.

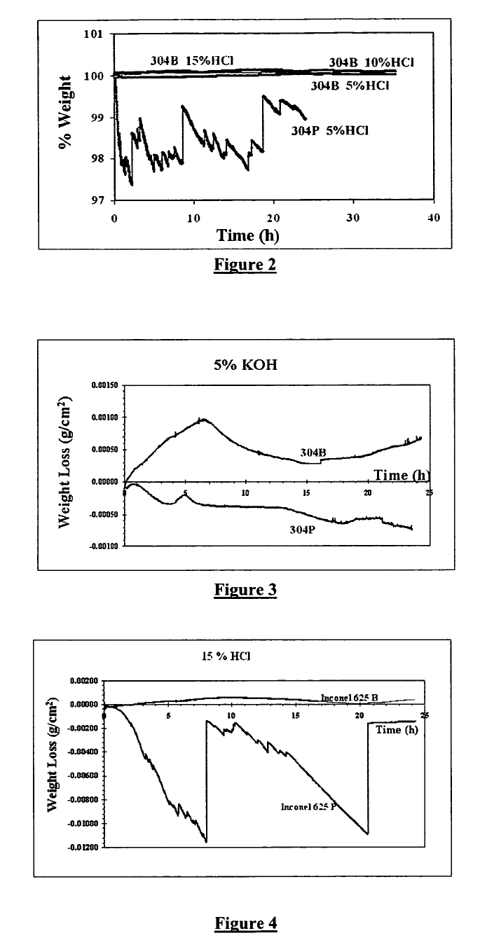

The results of the exemplary embodiments of the method described above are illustrated in FIG. 2, FIG. 3 and FIG. 4 as achieved by the presently disclosed system and method of use of said system. These continuous mass loss results were calculated as a function of time. FIG. 2 illustrates the deterioration resistance of boronized and unboronized AISI 304 specimens as evaluated in 5%, 10% and 15% HCl by the continuous mass loss technique presently disclosed. This figure is a representation of percent weight; as such the figure illustrates loss or gain of weight as compared to the original weight which is assigned to be a value of 100%. The results shown in FIG. 2 illustrate the fact that stainless steel has a weak resistance in hydrochloric acid. However, the boronized specimens of AISI 304 exhibited excellent resistance against deterioration in hydrochloric acid in the range of 5-15% HCl. From this method, the obtained oscillating graph of unboronized AISI304 indicated that unboronized or plain AISI 304 was repeated to form the passive film but it failed to form the passive film in hydrochloric acid. FIGS. 3 and 4 are graphic representations of the weight loss over time such that it shows a loss or gain of weight per unit surface area in real time. Accordingly, FIGS. 3 and 4 show the sensitivity of the presently disclosed system and method. FIG. 3 illustrates the deterioration resistance of boronized and unboronized AISI 304 specimens as evaluated in 5%, 10% and 15% KOH. The graphical representation illustrate that in 5% KOH, the boronized AISI 304 also showed better deterioration resistance than unboronized AISI 304. Finally, FIG. 4 illustrates the deterioration resistance of boronized and unboronized Inconel 625 in 15% HCl. Similar to boronized and unboronized AISI 304, the unboronized Inconel 625 showed the event of the forming film, which occurred in 1 second, while the boronized coating of Inconel 625 was able to prevent the substrate from the corrosive medium and reduce the deterioration rate of the specimen. These results illustrate the advantages and often necessity of being able to measure mass loss and subsequently deterioration rates in a second by second analysis.

Applicant has attempted to disclose all embodiments and applications of the disclosed subject matter that could be reasonably foreseen. However, there may be unforeseeable, insubstantial modifications that remain as equivalents. While the present invention has been described in conjunction with specific, exemplary embodiments thereof, it is evident that many alterations, modifications, and variations will be apparent to those skilled in the art in light of the foregoing description without departing from the spirit or scope of the present disclosure. Accordingly, the present disclosure is intended to embrace all such alterations, modifications, and variations of the above detailed description.

### REFERENCES

(1) “Standard Practice for Preparing, Cleaning, and Evaluating Corrosion Test Specimens” ASTM G1-90 (Re-approved 1999), ASTM International, West Conshohocken, Pa., 1999.
(2) “Standard Practice for Laboratory Immersion Corrion Testing of Metals” ASTM G31-72 (Re-approved 1999), ASTM International, West Conshohocken, Pa., 1999.

We claim:

1. A system to determine a deterioration rate of a material comprising:
a measuring device configured to measure a sample of the material at a time interval;
wherein the system is configured to expose the sample to a process environment; and
wherein the measuring device is coupled to the sample.

2. The system of claim 1, wherein the measuring device comprises a balance.

3. The system of claim 2, wherein the balance further comprises a hook.

4. The system of claim 1, wherein the measuring device measures the sample at a constant or variable time interval.

5. The system of claim 4, wherein the measuring device measures the sample at a continuous time interval.

6. The system of claim 1, further comprising a recording device configured to determine a deterioration rate of the sample.

7. The system of claim 1, further comprising a recording device configured to determine a deterioration rate of the sample at n intervals.

8. The system of claim 6, wherein the recording device comprises a computer.

9. The system of claim 6, wherein the recording device is an internal or external component of the measuring device.

10. The system of claim 1, further comprising a suspension device.

11. The system of claim 10, wherein the suspension device comprises a non-reactive entity.

12. The system of claim 10, wherein the suspension device comprises a platinum wire.

13. The system of claim 10, wherein the suspension device is configured to position the sample in the process environment.

14. The system of claim 10, wherein the suspension device comprises a wire.

15. The system of claim 10, wherein the suspension device further comprises a hook.

16. The system of claim 1, wherein the process environment comprises one or more zones of a gaseous environment, a liquid environment, a solid/particle flow environment, or a mixture of a gaseous environment and a liquid environment, a mixture of a gaseous environment and a solid/particle flow environment or a mixture of a liquid environment and a solid/particle flow environment.

17. The system of claim 1, wherein a temperature in the process environment is controllable.

18. The system of claim 1, wherein a pressure in the process environment is controllable.

19. The system of claim 1, wherein a moisture level in the process environment is controllable.

20. The system of claim 1, wherein a time interval in the process environment is controllable.

21. The system of claim 1, wherein a chemical combination in the process environment is controllable.

22. The system of claim 1, wherein the process environment comprises a corrosive substance.

23. The system of claim 1, further comprising a reflux condenser.

24. A method for calculating a deterioration rate of a sample, comprising:
positioning a sample within a process environment;
measuring a first weight at a first time interval;
measuring a nth weight of the sample at an nth time interval;
calculating the deterioration rate.
